(19) United States
(12) Patent Application Publication
(10) Pub. No.: US 2010/0086581 A1
(43) Pub. Date: Apr. 8, 2010

(54) METHOD FOR PURPURA REDUCTION AND PREVENTION

(76) Inventor: Ernest Bove, Fort Lauderdale, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 12/246,528

(22) Filed: Oct. 7, 2008

(51) Int. Cl. A61K 9/70; A61K 31/573; A61K 31/56

(52) U.S. Cl. 424/449; 514/179; 514/183

(57) ABSTRACT

A method for reducing or eliminating skin discoloration caused by purpura or similar conditions uses corticosteroids, particularly glucocorticoids, in a long term cutaneous treatment. A cream, ointment, spray or other means is used to apply a glucocorticoid, for example, hydrocortisone to warmed skin having a red or purple discoloration. Application is continued daily for several months or years to reduce, eliminate and prevent discoloration.

### CROSS-REFERENCE TO RELATED APPLICATIONS

None

### STATEMENT REGARDING FEDERALLY SPONSORED RESEARCH OR DEVELOPMENT

None

### REFERENCE TO SEQUENCE LISTING, A TABLE, OR A COMPUTER PROGRAM LISTING COMPACT DISC APPENDIX

None

### BACKGROUND OF THE INVENTION

1. Field of the Invention

The present invention relates to a method for treating skin discoloration caused by purpura. Specifically, this invention reduces or eliminates red discoloration of regions of skin.

2. Prior Art

Skin discoloration is one of many changes that accompany aging. It is common for people to develop moles, liver spots eczema and uneven skin tone as they age. While these changes are typically harmless, they are widely considered less attractive and may be disconcerting to an afflicted person.

Another common change in the skin is purpura. The term “purpura” is generally understood to mean the extravasation of “formed elements of the blood” from dermal blood vessels into the skin (for example dermis). In idiopathic thrombocytopenic purpura there is typically a tendency to bleed with hematoma formation (extravasation), which is crucially assisted by a depletion of blood platelets. An “inflammatory” purpura is frequently associated with vasculitis which may be caused by immune complexes. Some forms of purpura can be quite detrimental and require treatment with a variety of pharmaceuticals.

However, less severe types of purpura appear as regions of a person's skin that have turned a deep red and/or purplish color. This is caused by the leaking of blood elements into the dermis, becoming visible red blots. Such manifestations of purpura are harmless but undesirable. Patients with this condition are without recourse. Several treatments have been elucidated for redness caused by rash, inflammation, infection and other acute causes. However, permanent, chronic reddening of the skin has no cure or even means to reduce the discoloration.

U.S. Pat. No. 5,104,657 to Abdulla discloses a combination preparation for cutaneous application comprising antiseptic and antibiotic components and optionally cortisone. It is intended for treating difficult to heal skin disorders such as chronic eczema and inflammation of the anus. It does not offer any relief from permanent skin discoloration or damage from purpura.

U.S. Pat. No. 6,235,722 to Jayapathy discloses a hydrocortisone cream to treat fungal infections of endothelial tissues including the skin and ear canal. It does not offer or suggest any relief from permanent skin discoloration or damage from purpura.

U.S. Pat. No. 5,372,815 to Hodutu discloses a moisturizing cream for treating eczema and other inflammatory skin conditions. The invention reduces redness in that it reduces inflammation but does not suggest any means of altering permanently discolored skin.

U.S. Pat. No. 6,261,603 to McElwain discloses a skin cream having anti-aging properties. It includes a multitude of chemicals, compounds and extracts commonly known in the skin cream industry and purports to rejuvenate skin. However, it does not aid in restoring original skin tone to areas reddened by purpura.

U.S. Pat. No. 5,023,251 to Sattler et al. and U.S. Pat. No. 4,794,106 to Takashima et al. both disclose creams containing a hydrocortisone diester for treating inflamed skin. They do not offer or suggest any relief from permanent skin discoloration or damage from purpura.

U.S. Pat. No. 6,248,365 to Romisch et al. discloses a pharmaceutical preparation for treating inflamed endothelial cells, including cells of the intestinal wall. It does not offer or suggest any relief from permanent skin discoloration or damage from purpura.

Treatments for the mild forms of purpura have perhaps been somewhat slow in materializing simply because it is not harmful or deleterious. Many treatment plans have been developed for skin redness associated with the nimiety of afflictions involving skin infection, inflammation and irritation. However, purpura that only affects skin color has often been dismissed as one more unavoidable consequence of living long.

It is therefore desirable to provide a method for preventing, reducing or eliminating permanent red skin discoloration.

It is also desirable to provide a method for preventing, reducing or eliminating red skin discoloration resulting from purpura or similar conditions and not caused by inflammation or infection.

### BRIEF SUMMARY OF THE INVENTION

The present invention provides a means for reducing or eliminating permanent red or purplish discolorations of the skin induced by purpura or other causes, as is often experienced by persons as they age and is often found on the limbs. The invention comprises first applying hot water to the afflicted region of skin. It is important that the skin be warmed. A glucocorticoid cutaneous cream is then applied to the area. The glucocorticoid is gently massaged into the region of skin discoloration until the cream is completely absorbed and no longer visible. This process is repeated over several months. The treatment is typically continued as preventative maintenance to ensure that the discoloration does not return. The length of time required to remove the discoloration will depend on how significant the discoloration is. Typically the effect of the treatment will be noticeable after only a few months, but it will typically take a year or more to completely eliminate the discoloration.

The glucocorticoid cream is preferably used to apply glucocorticoid to the area of skin to be treated. It is also preferable to include various agents that enhance absorption of the glucocorticoid and its efficacy. For example, moisturizers such as aloe and aloe extracts may be included. Other agents to adjust the viscosity of the cream, such as parabens, polyethylene glycol, wax, paraffin, oil based polymers and/or gels may be included as well as emulsifiers and other agents. Preferably the glucocorticoid is blended into a cream having agents that aid the skin in absorbing the glucocorticoid such that it penetrates the epidermis and the dermis.

Only a small amount of glucocorticoid is required for the invention. The process as described above is performed once a day. Optionally, the glucocorticoid may be applied more than once a day. Increasing the frequency of application of the glucocorticoid will reduce the amount of time required for the invention to effectively reduce the discoloration. However, it will still take several months to remove discoloration.

Optionally, the method may be conducted utilizing a patch providing a slow release of the glucocorticoid. Typically, however, it is best to apply a cream after the skin has been heated. It is also preferred to massage a cream into the skin to aid in thorough absorption.

It is therefore an object of the invention to provide a method of reducing or eliminating skin discoloration by prolonged application of a glucocorticoid.

There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE DRAWINGS

FIG. 1 is a diagram of corticosterone.

FIG. 2 is a diagram of cortisol.

FIG. 3 is a diagram of dexethosone.

FIG. 4 is a diagram of the method of the invention.

### DETAILED DESCRIPTION OF THE INVENTION

The invention comprises the use of corticosterone derivatives, typically one or more glucocorticoids, to treat, reduce and eliminate red and purple skin discolorations. Although the precise mechanism is unknown, glucocorticoids are effective in reducing and eventually eliminating skin discolorations associated with purpura when applied to the affected region for a prolonged period.

The discolored region of a person's arm is typically dark red or purple in color, due to blood leaking into the dermis. This is known as purpura and was generally considered untreatable before now. Although the mechanism by which the invention operates is uncertain, the invention requires a glucocorticoid to be applied to the afflicted region after the region to be treated has been flushed with warm water. Preferably, the glucocorticoid is cortisol. Those skilled in the art will appreciate that hydrocortisone is a synthetically created cortisol and is therefore also equally preferred.

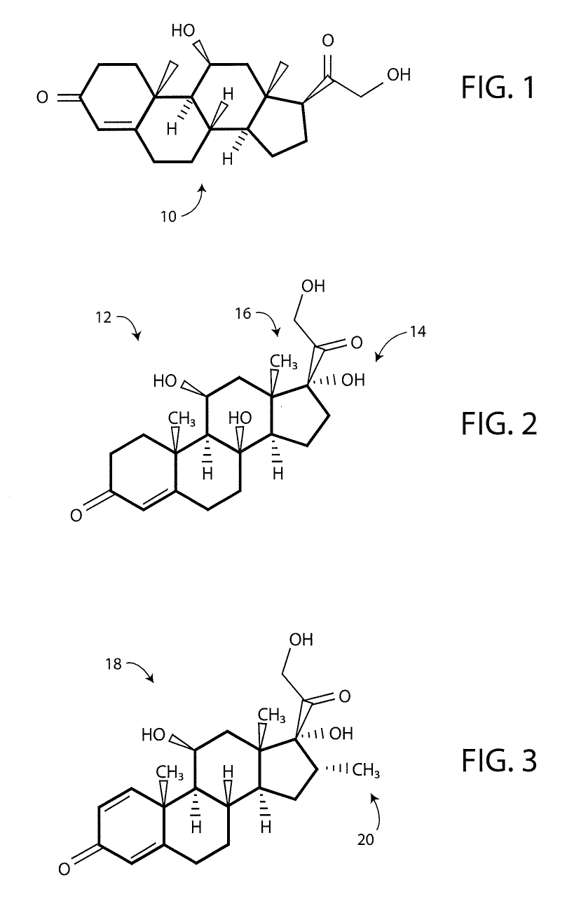

Glucocorticoids are a subclass of corticosteroids, all of which derive from corticosterone 10 shown in FIG. 1. Corticosteroids are known to have a variety of functions and glucocorticoids are those corticosteroids typically found to affect glucose metabolism. They also are known to influence the inflammatory response and cell-mediated immune responses. Whether the affect glucocorticoids have upon skin discoloration is related to some, all or none of these other activities is unknown. Corticosterone 10 is the main building block from which all glucocorticoids are formed.

And as used herein, cortisol and hydrocortisone refer to the same compound 12, shown in FIG. 2. cortisol 12 is the preferred glucocorticoid for use with the present invention. Cortisol 12 has the basic structure of its substrate corticosterone 10 plus hydroxyl group 14 and methyl group 16. Other glucocorticoids, such as dexamethasone 18 shown in FIG. 3, may also be used with the invention. Dexamethasone 18 has the same structure as cortisol 12 plus and added methyl group 20. These slight changes in structure affect the various activities of the compounds and therefore some glucocorticoids may be more suitable than others for the invention depending on a variety of factors. Because the invention involves heat and the method occurs over a long period of time, it is preferable to use glucocorticoids that are thermostable and very slow to degrade. Suitable glucocorticoids include, but are not limited to, cortisol, cortisone acetate, prednisone, prednisolone, methylprednisolone, dexamethasone, betamethasone, triamcinolone, beclometasone, fludrocortisone acetate, deoxycorticosterone acetate, and aldosterone. Those skilled in the art will appreciate that while cortisol is preferred, it belongs to the very large corticosteroid family and it is likely that many of the other members of the family either alone or in combination may be suitable for use with the invention.

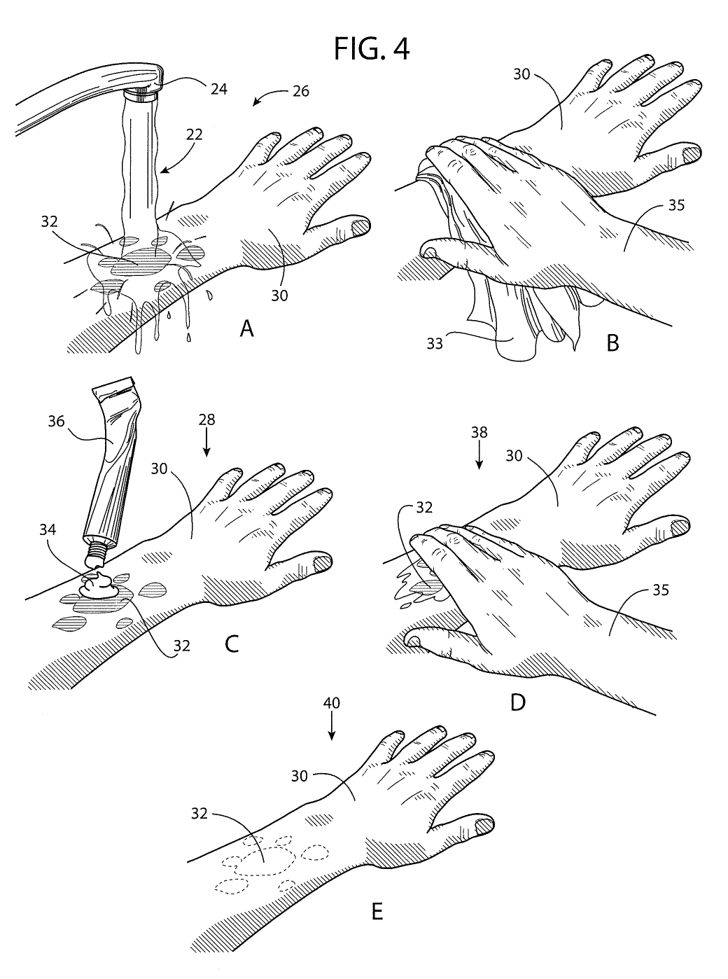

FIG. 4 shows the method of the present invention as applied to an afflicted person. In the first step 26, shown in FIG. 4A, hot water 22 is run from faucet 24 over discolored region 32 on arm 30. This is continued long enough to warm the skin of the discolored region. This has the effect of softening the skin and enhancing permeability of the epidermis. While this step may seem somewhat contra-indicated, as the cream is preferably oil-based and would reduce miscibility of the cream with the liquid aspects of the skin, it aids in the efficacy of the invention by working synergistically with the glucocorticoid in the cream. The water is preferably hot, but not at a temperature that will inflame or damage the skin. It is preferred to use water rather than hot air, as hot air may dry out the skin and fail to have a synergistic effect with the glucocorticoid.

FIG. 4B shows second step 37 in which the discolored region is dried. Towel 33 is applied using a person's other hand 35 to remove most of the moisture from arm 30. This also serves to clean the area to be treated. When completed, the discolored region to be treated is warm and very slightly moist, having absorbed moisture from the first step 26.

In the third step 28 shown in FIG. 4C, a glucocorticoid cream 34 is applied to the discolored region 32, which is now warm. The glucocorticoid cream 34 is dispensed onto the afflicted region 32 from a tube container 36. In this embodiment the cream 34 is applied directly to the afflicted region 32. However, it may be desirable to first place the cream onto a hand or washcloth to be used to massage in the cream first. Typically, only a small amount of the cream need be applied. This embodiment utilizes a glucocorticoid cream. However, the glucocorticoid may also be applied using a spray bottle, a mist, or even applied as a solid similar to a deodorant stick.

In the fourth step 38 shown in FIG. 4D, hand 35 is used to gently massage the glucocorticoid cream 34 into the discolored region 32. The cream 34 is rubbed into the discolored region 32 completely, until it has been fully absorbed. The massaging into the skin step 38 should be done gently so as not to irritate or damage the skin. When a solid stick is used, the glucocorticoid is applied to the discolored region and massaged into the skin simultaneously. This reduction in the number of steps may be advantageous for some persons.

Upon completion of step 38, arm 30 and discolored area 32 is left alone between treatments in step 40 shown in FIG. 4E. The process, beginning with the first step 26 is repeated several hours later or the following day. This is repeated daily, or more than once daily, for several months. The process may take over a year to substantially eliminate the discoloration. After the discoloration associated with purpura is removed, the process is continued, perhaps not as regularly, as a preventative measure so that the discoloration does not return.

One particularly well suited cream is an oil-based cream that includes the following ingredients:

### Active Ingredients Hydrocortisone (1%)

Inactive Ingredients Aloe Barbadensis Leaf Juice, Aluminum Sulfate, Beeswax, Calcium Acetate, Cetearyl Alcohol, Cetyl Alcohol, Cholecalciferol, Dextrin, Glycerin, Isopropyl Palmitate, Maltodextrin, Methylparaben, Mineral Oil, Petrolatum, Propylene Glycol, Propylparaben, Retinyl Palmitate, Sodium Cetearyl Stearate, Sodium Lauryl Sulfate, Tocopheryl Acetate, Water, Zea Mays Oil (Corn)

The above cream includes a moisturizer, aloe, a skin rejuvenator, tocopheryl acetate, emollients such as propylparaben, mineral oil and petrolatum, and a glucorticoid, hyrdrocortisone, also known as cortisol. Cortisol is readily available and highly susceptible to being incorporated into cutaneous creams. However, other corticosteroids and particularly other glucocorticoids may be used.

Only a relatively small amount of glucocorticoid is needed to practice the invention. This is preferred because a small amount of the glucocorticoid may absorb through the epidermis and into the blood stream, thereby negatively affecting metabolism. In the present invention, it is generally preferred that the glucocorticoid treats the dermis and not be absorbed into the blood stream. One of the salient features of the present invention is that glucocorticoid is not used to treat the blood itself but rather the epithelial cells of the skin. While blood may be the cause of the discoloration, the glucocorticoid in the present invention does not influence blood chemistry as it does when hydrocortisone is used to treat inflammations and infections, where it often is effective due to its influence on cell-mediated and other immunological responses. Those skilled in the art will appreciate that the use of glucocorticoids of the present invention is substantially different from known applications and therapies using cortisteroids and similar chemicals and represents a new area of corticosteroids application.

There are many hydrocortisone creams on the market used for treating rashes and itchy, inflamed skin. These are generally well suited for the invention. However, the glucocorticoid may be applied to the skin by any of several means including, but not limited to, creams, gels, lotions, ointments, tinctures, sprays, misters, patches, rub on sticks, wipes, washcloths and the like. Patches may be slow release patches similar to nicotine or birth control patches or other patches that expose skin to a chemical as are known in the art. When patches are used with the invention, it is not necessary to massage the glucorticoid into the skin as it will absorb slowly over time. Generally, creams, lotions and the like are preferred as they do not require an often undesirable patch to be constantly worn.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

The invention claimed is:

1. A method for improving skin coloration comprising:
a warming a discolored region of skin tissue of a person; and
b applying a glucocorticoid to the discolored region, such that the glucocorticoid is absorbed into the discolored region;
repeating steps a and b daily for more than ninety days.

2. The method of claim 1 wherein the warming of the discolored region of skin comprises running hot water over the discolored region and then drying the discolored region.

3. The method of claim 2 wherein the applying a glucocorticoid to the discolored region, such that the glucocorticoid is absorbed into the discolored region comprises applying a cream and massaging the cream into the discolored region.

4. The method of claim 3 wherein the cream is oil-based and comprises at least one moisturizer and wherein the glucocorticoid is selected from the group consisting of cortisol, cortisone acetate, prednisone, prednisolone, methylprednisolone, dexamethasone, betamethasone, triamcinolone, beclometasone, fludrocortisone acetate, deoxycorticosterone acetate, and aldosterone.

5. The method of claim 4 wherein the glucocorticoid comprises hydrocortisone.

6. The method of claim 4 wherein the glucocorticoid comprises dexamethasone.

7. The method of claim 4 wherein the cream comprises 1% hydrocortisone, aloe barbadensis leaf juice, aluminum sulfate, beeswax, calcium acetate, cetearyl alcohol, cetyl alcohol, cholecalciferol, dextrin, glycerin, isopropyl palmitate, maltodextrin, methylparaben, mineral oil, petrolatum, propylene glycol, propylparaben, retinyl palmitate, sodium cetearyl stearate, sodium lauryl sulfate, tocopheryl acetate, water, and corn oil.

8. The method of claim 2 wherein the applying a glucocorticoid to the discolored region, such that the glucocorticoid is absorbed into the discolored region comprises adhering a slow release patch to the discolored region, wherein the slow release patch exposes of the glucocorticoid to the discolored region so that the glucocorticoid is capable of being absorbed by the discolored region.

9. The method of claim 8 wherein the glucocorticoid comprises hydrocortisone.

10. The method of claim 8 wherein the glucocorticoid comprises dexamethasone.

11. The method of claim 2 wherein the applying a glucocorticoid to the discolored region, such that the glucocorticoid is absorbed into the discolored region comprises spraying a liquid including a glucocorticoid to the discolored region.

12. The method of claim 11 wherein the glucocorticoid comprises hydrocortisone.

13. The method of claim 11 wherein the glucocorticoid comprises dexamethasone.

14. The method of claim 1 wherein the applying a glucocorticoid to the discolored region, such that the glucocorticoid is absorbed into the discolored region comprises applying a cream and massaging the cream into the discolored region.

15. The method of claim 14 wherein the cream is oil-based and comprises at least one moisturizer and wherein the glucocorticoid is selected from the group consisting of cortisol, cortisone acetate, prednisone, prednisolone, methylprednisolone, dexamethasone, betamethasone, triamcinolone, beclometasone, fludrocortisone acetate, deoxycorticosterone acetate, and aldosterone.

16. The method of claim 1 wherein the applying a glucocorticoid to the discolored region comprises spraying a liquid comprising the glucocoriticoid onto the discolored region.

17. The method of claim 1 wherein the applying a glucocorticoid to the discolored region comprises applying a slow release patch onto the discolored region.

18. A method for improving skin coloration comprising:
a running hot water over the discolored region;
b. drying the discolored region;
c applying a cream comprising a glucocorticoid to the discolored region, such that the glucocorticoid is absorbed into the discolored region; and,
d. repeating steps a through c daily for about 90 days;
wherein the glucocorticoid is selected from the group consisting of cortisol, cortisone acetate, prednisone, prednisolone, methylprednisolone, dexamethasone, betamethasone, triamcinolone, beclometasone, fludrocortisone acetate, deoxycorticosterone acetate, and aldosterone.

19. The method of claim 18 wherein the cream comprises 1% hydrocortisone, aloe barbadensis leaf juice, aluminum sulfate, beeswax, calcium acetate, cetearyl alcohol, cetyl alcohol, cholecalciferol, dextrin, glycerin, isopropyl palmitate, maltodextrin, methylparaben, mineral oil, petrolatum, propylene glycol, propylparaben, retinyl palmitate, sodium cetearyl stearate, sodium lauryl sulfate, tocopheryl acetate, water, and corn oil.
